(19) United States
(12) Patent Application Publication
(10) Pub. No.: US 2019/0221298 A1
(43) Pub. Date: Jul. 18, 2019

(54) Diagnostic with recommendation tool for treatment with naturally occuring organic material

(71) Applicant: Aari Ruben, Tucson, AZ (US)

(72) Inventor: Aari Ruben, Tucson, AZ (US)

(21) Appl. No.: 15/732,962

(22) Filed: Jan. 17, 2018

(51) Int. Cl. G16H 20/10; G16H 70/40; G16H 10/60; G16H 50/20

(52) U.S. Cl. CPC G16H 20/10; G16H 50/20; G16H 10/60; G16H 70/40

(57) ABSTRACT

The invention provides a diagnostic system for naturally occurring organic material including cannabinoid, cannabidiol, and terpenoid treatment. A central computer utilizes a database containing treatment data relating to the use of naturally occurring organic material treatment of specific human conditions. Using patient generated information from a remote data entry apparatus, the central computer withdraws relevant treatment modalities and presents these treatments to the patient and treatment professional.

This is a continuation of U.S. patent application Ser. No. 14/120,232, filed on May 9, 2014, and entitled “Diagnostic with Recommendation Tool for Treatment with Naturally Occurring Organic Material”.

### BACKGROUND OF THE INVENTION

This invention relates generally to the use of cannabinoids and more particularly to a diagnostic aid therefore.

Cannabinoids are a class of diverse chemical compounds. Cannabinoids act on cells to repress neurotransmitter release in the brains. These receptors proteins include endocannabinoids which naturally occur in the body, phytocannabinoids found in cannabis and other plants, and synthetic cannabinoids which are man-made. There are at least eighty-five different cannabinoids which may be isolated from cannabis. These cannabis derived cannabinoids have a varied effect on the neurotransmitters and as such have varied treatment potentials.

Research into the use of cannabinoids for the treatment of a variety of diseases and conditions is reaching a feverish pace. It is quickly becoming clear that specific cannabinoids can have dramatic affect in improving the lives of many patients. This extends well past the treatment of nausea, glaucoma, or pain relief which has been traditionally treated with cannabis as a “shotgun” approach. It has become clear that the use of targeted cannabinoids for a specific ailment is much more effective.

The problem though is in the diagnostic capability which has heretofore evaded the medical community. The sheer amount of research which has been done quickly overwhelms the practitioner and the patient themselves cannot cope with the information.

It is clear there is a need for diagnostic tools for the effective use of cannabinoids.

### SUMMARY OF THE INVENTION

The present invention provides for a highly effective diagnostic system for plant based treatment. Those of ordinary skill in the art readily recognize a variety of plant based chemicals and combinations that are useful for the treatment of human health conditions, including, but not limited to cannabinoids, terpenoids, and other organic chemicals.

Within this discussion, cannabinoids will be used for illustration purposes but the invention is not intended to be limited only to cannabinoids. Other naturally occurring organic materials are also contemplated within the invention's purview.

In the preferred embodiment, a central computer utilizes a database memory containing treatment data relating to the use of cannabinoid treatment of specific human conditions. Information on the patient's condition is provided via a remote data entry apparatus.

Using the patient health condition data, the central computer accesses the database and withdraws selected treatment data from the database memory. Using this information, a treatment on the recommended cannabinoid treatment is generated and communicated to the patient.

As example, it has been found that certain cannabinoids are useful for the treatment of Multiple Sclerosis (MS). If the patient is suffering from MS, then these cannabinoids are useful and will be identified by the present invention.

While the invention ideally identifies specific cannabinoids, it is not intended to be so limited. The entire plant profile is often useful for certain types of treatment. As example, for Gastric Intestinal (GI) problems, it has been found that Cannabigerol (CBG) is a good treatment. Marijuana plants having high GSD content are then prescribed for GI problems.

Ideally, in order the facilitate the searching of the database by the central computer, the information therein is sorted using keyword data. With the search narrowed using the keyword data, the central computer is able to readily obtain the recommended cannabinoid treatment, the recommended mode of administering the preferred cannabinoid, a base reference article relating to the cannabinoid treatment; and, an abstract of the article.

In the preferred embodiment, input from the patient is guided using a decision tree. A decision tree guides the patient through a series of questions and as such eliminates redundant or irrelevant questions. Using past responses, the decision tree moves to narrow the possible recommended

All of this material is useful not only for searching purposes, but also to provide information to fully inform the patient of the treatment itself.

A further enhancement of the invention provides cost data associated with individual cannabinoids. In this way, the patient and the treating professional is able to budget and have more control over their treatment itself.

In some embodiments, the database is external to the central computer. This permits operators of the database to focus on maintaining an ever current set of references allowing the central computer to get a full data access to relevant articles.

In another embodiment, the central computer is not dedicated to a single remote apparatus but rather is able to handle multiple sources of patient input in a distributed system approach.

Record keeping is also important. This means that a record of what has been communicated to a particular patient is retained for later reference. Data identifying the patient is kept in a memory for later withdrawal and analysis.

The remote data entry used by the patient is any of a variety of apparatus well known to those of ordinary skill in the art, including, but not limited to electronic tablets, a patient computer, or a cellular telephone.

Another advantage of centralized computer system is that a record of recommended treatments permits the therapeutic potential to be refined and expanded. By retaining a history of recommended treatments, the treatments themselves are useful for the expansion and focus of the naturally occurring organic material. In the case of cannabis, hybrids can be created or the expanded production of certain strains of cannabis having desired levels of cannabinoids or terpenoids is possible.

Hybridization of a plant is well known to those of ordinary skill in the art, including, but not limited to those described in U.S. Pat. No. 7,982,109, entitled “Method for Improving Fertility of Hybrid Plants comprising Placing Fertility Restorer Genes into Multiple Gene Loci” issued to Knomori on Jul. 19, 2011; U.S. Pat. No. 5,929,307, entitled “Method for the Production of Hybrid Plants” issued to Hodges et al. on Jul. 27, 1999; U.S. Pat. No. 8,344,229, entitled “Argyranthemum Intergeneric Hybrid Plants and Method of Production” issued to Bernuetz on Jan. 1, 2013; U.S. Pat. No. 8,362,329, entitled “Interegeneric Hybrid Plants and Methods for Production Thereof” issued to Rooney et al. on Jan. 29, 2013; U.S. Pat. No. 8,112,936, entitled “Container-Based Plant Husbandry Apparatus and Controlled Horticultural Environment for Using Same” issued to Maslin et al. on Feb. 14, 2012; all of which are incorporated hereinto by reference.

The present invention also provides for a framework to improve the medical aspect of cannabis and other naturally occurring organic material. The database, which chronicles the recommendations being made, identifies which naturally occurring organic material has the greatest frequency of use. Through enhancement, the efficacy of that treatment is improved upon by further research or refinement.

The efficacy of the cannabidiols, cannabinoids, and/or terpenoids is accomplished through a variety of techniques such as hybridization of different strains of cannabis or by selective blending of oils from different strains to obtain a more tailored dosage.

Monitoring of databases with attendant reporting is well known in the art and include, but is not limited to, the techniques described in: U.S. Pat. No. 8,712,983, entitled “Database Management Method” issued to Yamada on Apr. 29, 2014; U.S. Pat. No. 8,719,274, entitled “Method, System, and Apparatus for Providing Generic Database Services within an Extensible Firmware Interface Environment” issued to Yakovlev on May 6, 2014; and, U.S. Pat. No. 8,719,276, entitled “Ranking Nodes in a Linked Database Based on Node Independence” issued to Haahr et al. on May 6, 2014; all of which are incorporated hereinto by reference.

This tool that the present invention provides is extremely powerful allowing for a focused refinement of the treatment.

The invention, together with various embodiments thereof, will be more fully explained by the accompanying drawings and the following description.

### DRAWINGS IN BRIEF

FIG. 1 is a block diagram illustrating the interactions found in the preferred embodiment.

FIG. 2 illustrates the preferred arrangement for the memory.

FIG. 3 is a flowchart of the preferred operation of the central computer.

FIG. 4 illustrates a decision tree used in one embodiment of the invention to identify proper treatment of a patient.

FIG. 5 is a flowchart of an embodiment used to recommend hybrid development of cannabis and other plants to obtain more efficient patient treatment.

### DRAWINGS IN DETAIL

FIG. 1 is a block diagram illustrating the interactions found in the preferred embodiment.

In this embodiment, the Internet 10 is used as a communication network. The invention is not intended to be limited to this network alone but includes those obvious to those of ordinary skill in the art including, but not limited to: WiFi types of networks, Local Area Networks, and Cellular Telephone systems.

Central computer 11 is accessible via Internet 10 and is able to draw data from database 12 using connections 17A and 17B. In some embodiments, database 12 is not remote from Central computer 11 but is accessible via link 16. As such, database 12 is optionally a remote database memory or is internal to central computer 11.

In the embodiment where database 12 is a remote database, other central computers 13A and 13B are able to operate and draw upon information using database 12 and links 17B/17C or 17B/17D. This embodiment of the invention permits one group to maintain the database while allowing many individual computers to provide the services described above.

Access to the central computer 10 is provided to the user via a variety of mechanisms well known to those of ordinary skill in the art, including, but not limited to user computer 14A and cellular telephone 14B. Tablets are also contemplated for this purpose.

As illustrated, user computer 14A accesses central computer 11 via the Internet 10 with links 17E/17A; cellular telephone 14B accesses the central computer 11 via links 17F/17A.

Once a report is ready for the user, it is communicated directly to the user's location of user computer 14A or cellular telephone 14B. Optionally, a printout via printer 15 using link 17G is also made for records.

Further records are ideally kept on database 12 including data identifying the user/patient as well as the recommended treatment. This record creates proper documentation and allows other computers to access the data for review and analysis. This database 12 then provides for an informational source for the selective enhancement of cannabis

Enhancement is accomplished when a summary (either electronically 17H or via printed report 171), ideally based on the frequency of treatment being prescribed, is provided to laboratory 18. The summary allows the laboratory to identify which treatments are most common and to target the production of the most useful naturally occurring organic.

As noted earlier, cannabis has a wide variety of components including cannabidiols, cannabinoids, and terpenoids. The enhancement provided by the laboratory is done through a variety of ways well known to those of ordinary skill in the art, including the hybridization of cannabis via greenhouse 19A which employs different strains of cannabis to obtain the desired levels of cannabidiols, cannabinoids, and/or terpenoids for the targeted patient treatment.

Alternatively, oils (cannabidiols, cannabinoids, and terpenoids oils) are extracted 19B and then mixed to obtain the desired concentrations for specific treatments.

This aspect of the invention, or tool, allows the medical cannabis industry to improve and enhance itself.

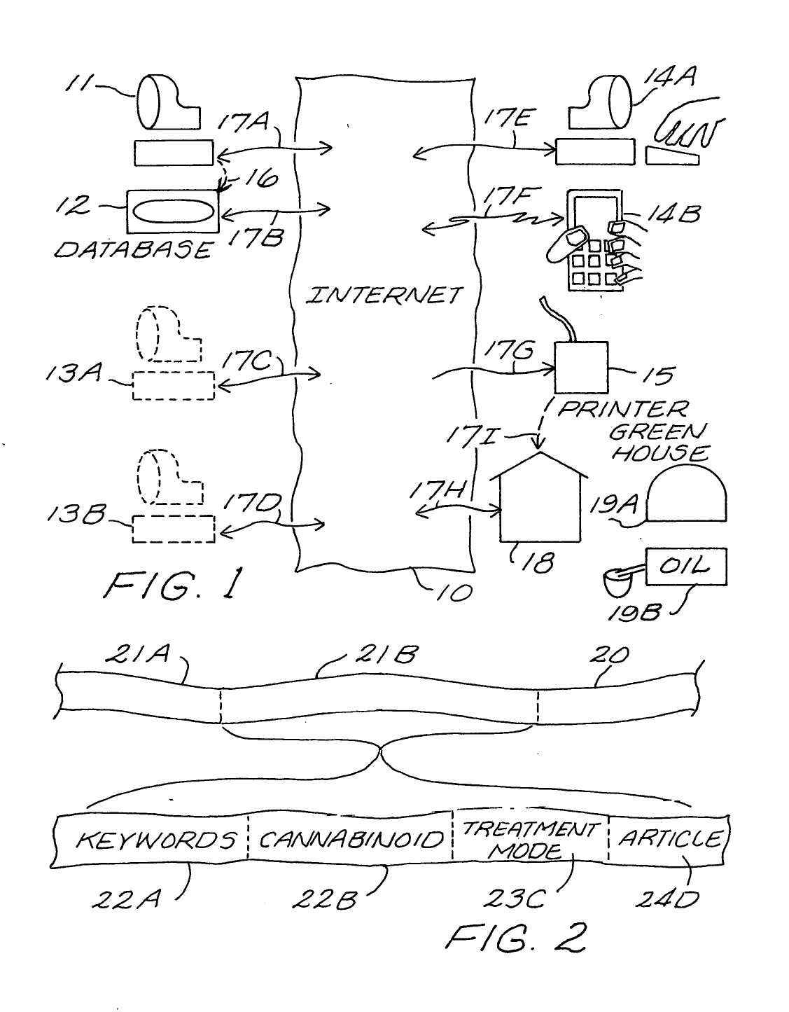

FIG. 2 illustrates the preferred arrangement for the memory.

Memory 20 is made up of memory segments 21A, 21B, etc. Each segment, as illustrated by 21B, has different blocks of data. The first block of data is a listing of the keywords 22A associated with the article. As example, the keywords could include: nausea, pain from cancer, chronic pain, etc.

The next block 22B contains a list of the cannabinoids which are recommended for treatment associated with the keywords. This block 22B is followed by a treatment mode 23C for the cannabinoids in block 22B. Those of ordinary skill in the art recognize a variety of treatment modes available, including, but not limited to: inhalation and digestion.

The final block 24D is an abstract of the article and the full article itself. Block 24D permits the patient or care-giver to educate themselves on the treatment by reading the entire article or the abstract.

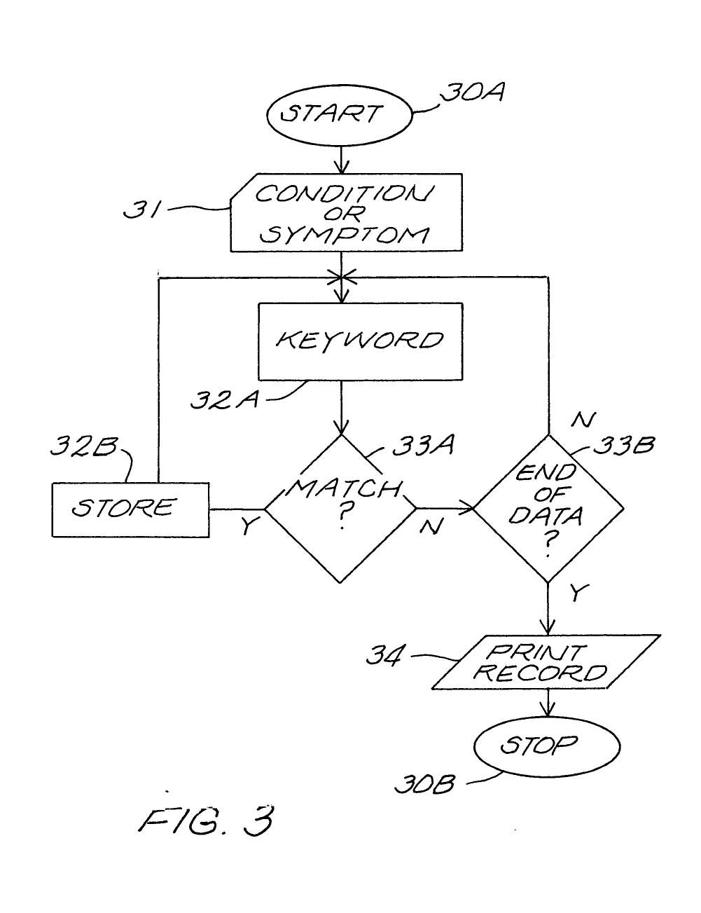

FIG. 3 is a flowchart of the preferred operation of the central computer.

Once the program starts 30A, the program receives data on the conditions or symptoms 31 that the patient is experiencing. A record from the database is obtained 32A with the keywords associated with the record. The record's keywords are then compared with the conditions or symptoms data to see if there is a match 33A.

If a match occurs, the relevant record is stored 32B in a volatile memory and the next record's keyword is obtained 32A.

If a match 33A, does not occur, a check on if the end of data has occurred 33B. If more records are available, then a new keyword is obtained 32A; otherwise, the stored material from the volatile memory is printed out 34 and the program stops 30B.

In some embodiments of the invention, instead of a printout 34, the results of the search are displayed or alternatively are communicated to the remote input apparatus used by the patient.

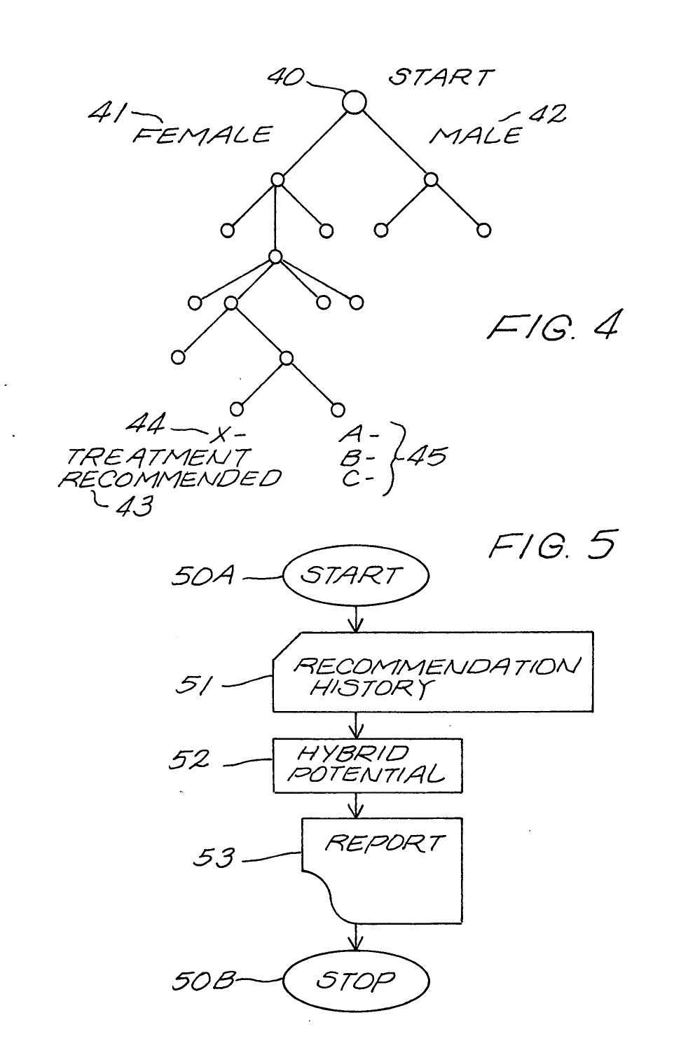

FIG. 4 illustrates a decision tree used in one embodiment of the invention to identify proper treatment of a patient.

In many applications, the use of a decision tree to guide the patient is ideal since a decision tree typically eliminates redundant or irrelevant questions directed to the patient. Using past responses, the decision tree moves to narrow the possible recommended treatments with each query/answer.

Typically a decision tree is illustrated as a mathematical graph having nodes (representing the question) and arc (identifying the response or answer).

As shown in FIG. 4, once start 40 begins, the patient is asked to identify if they are female 41 or male 42. This response in itself, eliminates many maladies, conditions, or diseases that the patient can have. As example, a male cannot be pregnant, a female cannot have testicular cancer.

Subsequent question may deal with such questions as age, past diseases, past injuries, etc. All of these questions move the patient through the decision tree to arrive at the final recommended treatment 43.

In this illustration, the final treatment 43 is one of two different approaches. One approach uses a single cannabinoid (or other naturally occurring organic material) 44; while a second recommended treatment uses a combination of three different naturally occurring organic materials 45 (A, B, and C).

In the case of treatment 45, the three naturally occurring organic materials can be taken in a variety of combinations obvious to those of ordinary skill in the art, including, but not limited to simultaneously, sequentially, or in the alternative. Additionally, if the dosage requires any special treatment such as titration schedules or if the dosage is to change over time, the report given to the patient provides these details.

As example, while the patient may have Multiple Sclerosis, use of certain cannabinoids would be useful for the treatment of muscle spasms or nerve pain.

FIG. 5 is a flowchart of an embodiment used to recommend hybrid development of cannabis and other plants to obtain more efficient patient treatment.

By retaining a history of recommended treatments, the treatments themselves are useful for the expansion and focus of the naturally occurring organic material. In the case of cannabis, hybrids can be created or the expanded production of certain strains of cannabis having desired levels of cannabinoids or terpenoids is possible.

The program starts 50A and obtains the history of recommended treatments 51 from the data base associated with the central computer. Using this historical data, the data is analyzed to determine which treatments are good recommendations for hybridization of cannabis so that the desired plant is obtained. This recommendation is given to the producer 53 who is then able to focus their growing or refining process to meet the current demands for the most prevalent treatments.

The program then stops 50B.

It is clear that the present invention provides for a highly improved tool for the effective use of cannabinoids, terpenoids, and other naturally occurring organic materials.

What is claimed is:

1. A diagnostic system comprising:
a) a central computer having a database memory containing treatment data relating to the use of naturally occurring organic materials used for treatment of specific human conditions;
b) a reporting computer collecting data on past treatment data of specific human conditions from a patient, said reporting computer communicating said past treatment data to the central computer for storage in said database memory;
c) a remote data entry apparatus receiving patient health condition data from a patient and communicating the patient health condition data to said central computer; and,
d) wherein said central computer,
1) withdraws selected past treatment data from said database memory based upon said patient health condition data, and,
2) communicates said selected past treatment data to the patient.

2. The diagnostic system according to claim 1, wherein said naturally occurring organic material is chosen from the group consisting of cannabidiols, cannabinoids, and terpenoids.

3. The diagnostic system according to claim 1, wherein for each treatment data within said database memory includes:
a) keyword data;
b) recommended naturally occurring organic material treatment;
c) recommended mode of administering said naturally occurring organic material;
d) an article relating to the naturally occurring organic material treatment; and,
e) an abstract of said article.

4. The diagnostic system according to claim 3, wherein said central computer communicates the recommended naturally occurring organic material treatment, the recommended mode of administering said naturally occurring organic material, and an abstract to the patient.

5. The diagnostic system according to claim 4, wherein,
a) said database memory further includes cost data associated with the naturally occurring organic material; and,
b) said central computer communicates selected cost data to the patient based upon the recommended naturally occurring organic material treatment.

6. The diagnostic system according to claim 5, wherein said data base is remote from said central computer; and wherein said central computer communicates with said data base via an Internet.

7. The diagnostic system according to claim 5, wherein said central computer logs said recommended naturally occurring organic material with data identifying said patient in a memory.

8. The diagnostic system according to claim 1, wherein said remote data entry apparatus includes an electronic tablet and wherein said central computer communicates with the patient via said electronic tablet.

9. The diagnostic system according to claim 1, wherein said remote data entry apparatus includes a patient computer and wherein said central computer communicates with the patient via said patient computer.

10. The diagnostic system according to claim 1, wherein said remote data entry apparatus includes a cellular telephone and wherein said central computer communicates with the patient via said cellular telephone.

11. A diagnostic system for cannabinoid treatment comprising:
a) a computer having a database memory containing treatment data relating to the use of cannabinoids for treatment of specific human conditions, wherein said computer: receives patient health condition data, withdraws selected treatment data from said database memory based upon said patient health condition data, and, prepares a recommended cannabinoid treatment; and,
b) Based upon the recommended cannabinoid treatment, a chemist prepares a cannabinoid complying with the recommended cannabinoid treatment.

12. A diagnostic system for cannabinoid treatment comprising:
a) a central computer;
b) a remote database memory containing treatment data relating to the use of cannabinoids treatment of specific human conditions;
c) at least two remote data entry apparatus operated by patients, one of said at least two remote data entry apparatus receiving patient health condition data from a user-patient, said remote data entry apparatus communicating the patient health condition data to said central computer; and,
wherein said central computer,
1) links with said remote database memory and withdraws selected treatment data from said remote database memory based upon said patient health condition data,
2) prepares a recommended cannabinoid treatment, and,
d) based upon the recommended cannabinoid treatment, a chemist prepares a cannabinoid complying with the recommended cannabinoid treatment.
